(19) United States
(12) Patent Application Publication
(10) Pub. No.: US 2024/0347206 A1
(43) Pub. Date: Oct. 17, 2024

(54) EARLY DETECTION OF CONDITIONS AND/OR EVENTS

(71) Applicant: Bayer Aktiengesellschaft, Leverkusen (DE)

(72) Inventors: Steffen VOGLER, Leverkusen (DE); Johannes Hohne, Leverkusen (DE); Matthias Lenga, Leverkusen (DE)

(73) Assignee: Bayer Aktiengesellschaft, Leverkusen (DE)

(21) Appl. No.: 18/602,895

(22) Filed: Mar. 12, 2024

(30) Foreign Application Priority Data
Mar. 13, 2023 (EP) 23161513.9

(51) Int. Cl. G16H 50/30; G16H 50/80

(52) U.S. Cl. CPC G16H 50/30; G16H 50/80

(57) ABSTRACT

The present disclosure relates to the early detection of the presence or occurrence of a condition in an object of investigation and/or the occurrence of an event in an object of investigation by means of machine learning methods. The subject matter of this disclosure consists of a computer-implemented method, a computer system, and a computer program for the early detection of such conditions and/or events.

### CROSS REFERENCE TO RELATED APPLICATIONS

This application claims the benefit of European Application No.: 23161513.9, filed on Mar. 13, 2023, the disclosures of which is herein incorporated by reference in its entirety.

### FIELD

The present disclosure relates to the early detection of the presence or occurrence of a condition in an object of investigation and/or the occurrence of an event in an object of investigation by means of machine learning methods. The subject matter of this disclosure consists of a computer-implemented method, a computer system, and a computer program for the early detection of such conditions and/or events.

### BACKGROUND

Predictions are encountered in a variety of ways in everyday life. There are predictions about the weather, climate, crop yields, damage caused by plant pests, on the course of a disease in a patient, the stock market value of a company and many other topics.

Predictions can help people prepare for conditions and/or events that may occur or arise in the future. Knowing that a condition may arise and/or an event may occur, measures can be taken to influence the effects associated with the occurring condition and/or event. Where appropriate, it is possible to prevent or delay the occurrence of a condition and/or an event if it is an undesirable condition and/or event.

One example is the occurrence or presence of a disease in a person.

There are a large number of diseases that can occur in a person over the course of a lifetime and which can mean a serious detriment and/or reduction in the quality of life for those affected.

Examples of such diseases are diabetes, asthma, allergies, atopic dermatitis, migraine, rheumatism, hypertension, depression, Alzheimer's, dementia, Parkinson's disease, cancer, chronic bowel inflammatory disease, endometriosis, age-related macular degeneration, etc.

Some diseases are associated with a predisposition, i.e. a person's susceptibility to the disease developing over the course of their life. This can be a genetic disposition or an acquired disposition.

For the people affected, early detection of diseases is beneficial, as the affected people can adapt to the disease on the one hand, and, on the other hand, measures can be taken at an early stage to prevent the outbreak of the disease, to delay its occurrence, to stop or slow down its progress and/or to alleviate symptoms.

Another example is the crop yield in a field used for growing crops. For a farmer, it may be important to know what crop yield the farmer can achieve by planting a particular crop variety in a field. In addition to environmental conditions such as temperatures, precipitation, sunlight and pest infestation, as well as measures taken by the farmer such as irrigation, fertilization and application of pesticides, the characteristics of the plant variety can also have an impact on crop yields. The farmer may be interested in predicting what yield can be achieved for a plant variety in a field, for example, to select the plant variety that promises the highest yield.

Another example is the wear of components in a machine or plant. It may be advantageous for a user of the machine or plant to find out which component is most likely to become worn and will need to be replaced and/or when a component will lose its functionality. It may be the case that a premature failure of a component has already been determined in the component.

There is therefore a need for early detection of the presence, occurrence and/or incidence of conditions and/or events.

### SUMMARY

The early detection of the existence, occurrence and/or incidence of conditions and/or events is made possible by the subject matter of the independent claims. Preferred embodiments are found in the dependent claims, the present description and the drawings.

A first subject matter is a computer-implemented method comprising the steps of:
receiving property data for an object of investigation,
receiving relational data, wherein the relational data comprises property data for one or more objects that are related to the object of investigation, and data on a relationship between the one or more objects and the object of investigation,
generating a numerical representation on the basis of the property data for the object of investigation and the relational data,
supplying the numerical representation to a trained machine learning model, wherein the trained machine learning model was trained on the basis of training data to determine an expected value for the presence and/or occurrence and/or incidence of a condition and/or event, wherein the training data for each reference object of a multiplicity of reference objects comprises i) property data for the reference object and ii) relational data comprising property data for other reference objects that are related to the reference object, and data relating to the relationships of the reference object to the other reference objects, and (iii) information on the presence and/or occurrence and/or incidence of the condition and/or event in the reference object,
receiving an expected value as the output from the trained machine learning model, wherein the expected value indicates a probability that the condition and/or event is present and/or will arise and/or will occur in the object of investigation,
outputting the expected value and/or storing the expected value and/or transmitting the expected value to a separate computer system.

The present disclosure further provides a computer system comprising:
an input unit
a control and calculation unit, and
an output unit,
wherein the control and computation unit is configured
   to cause the input unit to receive property data for an object of investigation and relational data, wherein the relational data comprises property data for one or more objects that are related to the object of investigation, and data on a relationship between the one or more objects and the object of investigation,
   to generate a numerical representation on the basis of the property data for the object of investigation and the relational data,
   to supply the numerical representation to a trained machine learning model, wherein the trained machine learning model was trained on the basis of training data to determine an expected value for the presence and/or occurrence and/or incidence of a condition and/or event, wherein the training data for each reference object of a multiplicity of reference objects comprises i) property data for the reference object and ii) relational data comprising property data for other reference objects that are related to the reference object, and data relating to the relationships of the reference object to the other reference objects, and iii) information on the presence and/or occurrence and/or incidence of the condition and/or event in the reference object,
   to receive an expected value from the trained machine learning model, wherein the expected value indicates a probability that the condition and/or event is present and/or will occur in the object of investigation,
   to cause the output unit to output and/or to store the expected value and/or to transmit it to a separate computer system.

A further subject matter of the present disclosure is a non-volatile computer-readable storage medium on which software commands are stored which, when executed by a processor of a computer system, cause the computer system to execute the following:
receiving property data for an object of investigation,
receiving relational data, wherein the relational data comprises property data for one or more objects that are related to the object of investigation, and data on a relationship between the one or more objects and the object of investigation,
generating a numerical representation on the basis of the property data for the object of investigation and the relational data,
supplying the numerical representation to a trained machine learning model, wherein the trained machine learning model was trained on the basis of training data to determine an expected value for the presence and/or occurrence and/or incidence of a condition and/or event, wherein the training data for each reference object of a multiplicity of reference objects comprises i) property data for the reference object and ii) relational data comprising property data for other reference objects that are related to the reference object, and data relating to the relationships of the reference object to the other reference objects, and (iii) information on the presence and/or occurrence and/or incidence of the condition and/or event in the reference object,
receiving an expected value as the output from the trained machine learning model, wherein the expected value indicates a probability that the condition and/or event is present and/or will arise and/or will occur in the object of investigation,
outputting the expected value and/or storing the expected value and/or transmitting the expected value to a separate computer system.

### BRIEF DESCRIPTION OF THE FIGURES

The following figures show various systems and methods, according to some embodiments.

FIG. 1 shows a graph, according to some embodiments.

FIG. 2A shows another graph, according to some embodiments.

FIG. 2B shows another graph, according to some embodiments.

FIG. 3A shows an adjacency matrix for the graph shown in FIG. 1, according to some embodiments.

FIG. 3B shows another adjacency matrix for the graph shown in FIG. 1, according to some embodiments.

FIG. 4 shows a machine learning model comprising a graph neural network, according to some embodiments.

FIG. 5 shows an autoencoder, according to some embodiments.

FIG. 6 shows a schematic for using augmentation techniques, according to some embodiments.

FIG. 7 shows an encoder combined with the machine learning model shown in FIG. 4, according to some embodiments.

FIG. 8 shows a method for training a machine learning model, according to some embodiments.

FIG. 9 shows a method for using a trained machine learning model, according to some embodiments.

FIG. 10 shows a computer system, according to some embodiments.

FIG. 11 shows another computer system, according to some embodiments.

### DETAILED DESCRIPTION

The provided techniques will be more particularly elucidated below without distinguishing between the subjects of the disclosure (method, computer system, computer-readable storage medium). Rather, the following embodiments shall apply mutatis mutandis to all aspects of the disclosure, irrespective of the context in which they occur (method, computer system, computer-readable storage medium).

Where steps are stated in an order in the present description or in the claims, this does not necessarily mean that the disclosure is limited to the order stated. Instead, it is conceivable that the steps can also be executed in a different order or else in parallel with one another, unless one step builds on another step, which absolutely requires that the step building on the previous step be executed subsequently (which will however be clear in the individual case). The orders stated are therefore preferred embodiments of the present disclosure.

In certain places the disclosure will be more particularly elucidated with reference to drawings. The drawings show specific embodiments having specific features and combinations of features, which are intended primarily for illustrative purposes; the disclosure is not to be understood as being limited to the features and combinations of features shown in the drawings. Furthermore, statements made in the description of the drawings in relation to features and combinations of features are intended to be generally applicable, that is to say transferable to other embodiments too and not limited to the embodiments shown.

The present disclosure describes means for early detection of the presence, occurrence and/or incidence of a condition and/or event in an object of investigation.

In one embodiment of the present disclosure, the object of investigation is a living being, preferably a plant, an animal, a human being or other organism.

However, the object of investigation may also be a field for crops, a plant variety, a part of a plant, a group of people, a part of a human being (e.g., an organ or multiple organs), a machine, a component, or another object or part of an object or a group of objects.

If the object of investigation is an animal or a human being, the object of investigation is also referred to as an individual or a patient in this description. A human being is also referred to as a person in this description.

In the case of a plant, an animal or human being, the condition and/or event may be an existing or occurring disease.

For a plant or group of plants or a field, the condition and/or event may be a crop yield. “Crop yield” is intended to mean the quantity of plant products present (e.g., expressed as total mass) at the time of harvesting.

In the case of a component, the condition and/or event may be a loss and/or a reduction in the functionality of the component.

In a first step, property data for the object of investigation is received.

The term “receive” encompasses both the reading and/or retrieving of data and the accepting of data transmitted for example to the computer system of the present disclosure. The property data (as well as the relational data) can be read from one or more data stores and/or transmitted by one or more separate computer systems and/or sensors and/or other devices.

Property data characterizes an object (e.g., the object of investigation). The object can be characterized by certain features. In the case of a person, these features include, for example, age, height, weight, body mass index (BMI), gender, eye colour, hair colour, skin colour, blood group, membership of an ethnic group, existing diseases and/or conditions, pre-existing diseases and/or conditions and/or similar properties. A picture (such as a photograph or medical image) showing the human body or part of it is an example of property data in the form of a collection of properties that characterize the person.

Other examples of property data for human beings include native language, membership of a religion, marital status, nationality, date of birth, place of birth (e.g., country and/or region and/or city), level of education, employment, level of income, wealth, debt, creditworthiness, place of residence (e.g., country, and/or city and/or postal code), living family members and others.

In the case of a human being or animal, the property data preferably includes health data. Health data are all data that can provide information about a person's or an animal's state of health.

In addition to the property data already mentioned, the following additional property data can also provide information about the health status of a human or animal: history of previous illnesses, times when the diseases occurred, severity of the diseases that occurred, measures taken to cure and/or alleviate the diseases, current and/or past blood tests (e.g., blood sugar, oxygen saturation, red blood cell count, haemoglobin content, white blood cell count, platelet count, inflammatory values, blood lipids), liver values, kidney values, thyroid values, blood pressure, resting heart rate, lung capacity, tidal volume, respiratory volume, internal body temperature, electrocardiogram, electroencephalogram, skin conductivity, tremor (frequency), amount and frequency of medication taken, amount and frequency of drugs taken, such as cigarettes and/or alcohol.

The property data may also include medical images.

A medical image is a visual representation of the human body or part of it or of the body of an animal or part of it. Medical images can be used for diagnostic and/or treatment purposes, for example.

Medical imaging techniques include X-ray, computed tomography (CT), fluoroscopy, magnetic resonance imaging (MRI), ultrasound, endoscopy, elastography, tactile imaging, thermography, microscopy, positron emission tomography, optical coherence tomography (OCT) and others.

Examples of medical imaging include CT scans, X-ray images, MRI scans, fluorescence angiography images, OCT scans, histopathological images, ultrasound images and others.

The property data may also include audio recordings, e.g., recordings of a person coughing, sneezing, snoring, inhaling and/or exhaling and/or speaking or of a person with hiccoughs, heartbeat sounds, gastrointestinal sounds, blood flow sounds and/or the like.

Human property data may include data derived from the person him/herself (e.g., self-assessment data). In addition to objectively recorded anatomical, physiological and/or physical data, the well-being of a person plays an important role in assessing their health. Subjective perception can also make a significant contribution to the understanding of objectively recorded data and the relationships between different data items. If, for example, sensors detect that a person has experienced physical stress, for example because the respiratory rate and heart rate have increased, this may be due to the fact that even a small amount of physical effort in everyday life puts a strain on the person; another possibility is that the person has consciously and willingly brought about the situation of physical stress, for example in the context of a sporting activity. A self-assessment can provide clarity here as to the causes of physiological anomalies. Thus, the question of self-assessment also plays an important role in clinical trials. The English-language literature uses the term “patient reported outcome” (PRO) as a generic term for many different concepts for the measurement of subjectively perceived states of health. The common basis for these concepts is that the patient's status is personally assessed and reported by the patient. The subjective perception can be measured by means of a computer system, which can be used by the patient to record information about the subjective health status. For example, a list of questions to be answered by the patient can be used. Preferably, the questions are answered with the aid of a computer system (e.g., a tablet computer or a smartphone). One possibility is that the patient is shown questions displayed on a screen and/or read out via a speaker. One possibility is that the patient enters the answers to the questions into a computer system by entering text via an input device (e.g. keyboard, mouse, touch screen and/or microphone (via voice input)) or by selecting and/or marking predefined answers and/or answer fields. It is conceivable that a chatbot is used in order to facilitate the input of all items of information for the patient. It is conceivable that the questions are recurring questions to be answered by a patient once or multiple times a day. It is conceivable that some of the questions are asked in response to a specific event. It is, for example, conceivable that it is captured by means of a sensor that a physiological parameter is outside a defined range (e.g., an increased respiratory rate is detected). As a response to this event, the patient can, for example, receive a message via his/her smartphone or a smartwatch or the like that a defined event has occurred and that said patient should please answer one or more questions, for example in order to find out the causes and/or the accompanying circumstances in relation to the event. The questions can be of a psychometric nature and/or preference based. At the heart of the psychometric approach is the description of the external, internal and anticipated experiences of the individual, by the individual. Said experiences can be based on the presence, the frequency and the intensity of symptoms, behaviours, capabilities or feelings of the individual questioned. The preference-based approach measures the value which patients assign to a health status.

Features of an object of investigation can be described and/or captured and/or stored and/or processed in various ways, also known as modalities.

For example, the result of a pregnancy test can be represented by an image of the test unit with a specific colour that indicates the test result, or it can be represented by a line of text that reads: “This person is pregnant.” It can also be a circle with a check mark at a specific point on a structured form. It can also be the number 1 as opposed to the number 0 in the memory of a computer system, electronic device, server or the like. All of these data types contain the same information, but in the form of different representations/modalities.

Common modalities are numbers, text, images, and audio recordings. The present disclosure is not limited to a specific modality. How different modalities can be processed is described later in this description.

In addition to the property data of the object of investigation, relationship data is received. Relationship data (also known as relational data) includes property data for one or more other objects that are related to the object of investigation. Relations can be precisely defined or ambiguously defined. The relational data also includes data on the relationship between the object of investigation and the one or more other objects.

Typically (but not necessarily), the at least one object to which the object of investigation is related is of the same type (but preferably not the exact same) object as the object of investigation. If the object of investigation is a human being, the at least one object is preferably also a human being, preferably another human being. If the object of investigation is an animal, the at least one object is preferably also an animal, preferably another animal of the same species. If the object of investigation is a plant, the at least one object is preferably also a plant, preferably another plant of the same variety or a plant of another variety but of the same species. If the object of investigation is a field, the at least one object is preferably also a field, preferably another field on which the same crops are grown or the same field at another time (e.g., at a time during a past vegetation period). If the object under investigation is a component or a machine, the at least one object is preferably also a component or a machine, preferably of the same type and from the same or another production batch.

The terms “relation” and “relationship” describe how the at least one object and the object of investigation are related to each other and are used synonymously in this description. The terms “relation” and “relationship” can indicate what the object of investigation and the at least one object have in common and/or how they differ from each other and/or how they interact with each other and/or how they depend on each other and/or are descended from each other.

For example, for living beings, the relationship can indicate the kinship relationship. For example, if the object of investigation is a person, there may be one or more other persons who have a defined kinship relationship to the person of interest, such as a mother, a father, one or more siblings, two grandmothers, two grandfathers, possibly one or more maternal and/or paternal aunts and/or uncles of first, second and/or higher degree, possibly one or more maternal and/or paternal female and/or male cousins at one, two and/or higher remove, possibly one or more children and grandchildren, one or more nieces and/or nephews of first, second and/or higher degree, and/or possibly one or more great nieces and/or great nephews of first, second and/or higher degree and/or other possibly other relatives. This applies similarly to animals and plants that sexually reproduce.

The property data for the one or more objects to which the object of investigation is related characterizes the one or more objects. This is preferably the same data as the property data for the object of investigation. This means that if there is data on the age, size and weight of the object of investigation for the object of investigation, there is preferably also data on the age, size and/or weight of one or more objects that is/are related to the object of investigation.

However, it should be noted that all data that exists for the object of investigation does not have to exist for every object. If there are multiple objects related to the object of investigation, the same data does not need to exist for all objects; some data may be missing. Conversely, there may be data for one or more objects that do not exist for the object of investigation. As a general rule, the more data is available for the object of investigation and the at least one object, the more accurate predictions can be made about the presence and/or occurrence and/or incidence of a condition and/or event.

In a further step, a numerical representation is created on the basis of the property data for the object of investigation and the relational data.

The numerical representation represents the object of investigation with its properties and in its relation to an object with its properties or to multiple objects with their properties in the form of numbers and/or arrays of numbers.

An array of numbers may be a tuple, list, vector, matrix, tensor, or other array of numbers and/or include one or more of the above arrays.

If property data is not present in numerical form (e.g. as text), it must be converted into a numerical form.

For example, categorical features can be (arbitrarily) assigned numbers. The use of 1-of-n codes (one-hot encodings) for non-numerical features is possible.

In a preferred embodiment, the numerical representation is a graph. A graph is a structure that represents a set of objects along with the connections (relationships) between these objects. The mathematical abstractions of the objects are called nodes (also corners) of the graph. The pairwise connections between nodes are called edges (sometimes arcs) and represent the relationships between the objects.

Thus, a graph in the sense of the present disclosure comprises a node representing the object of investigation (with its properties). The graph also includes an additional node for each additional object for which property data exists and which is related to the object of investigation. In other words, each further node of the graph represents a further object (with its properties) that is related to the object of investigation. The edges represent the relationships. An edge between the object of investigation and another object represents the relationship between the object of investigation and the other object.

FIG. 1 shows an example of a graph. The graph includes a node in the form of a blacked-out circle representing the object of investigation U with its properties. The graph also includes further nodes in the form of white circles representing the further objects O1, O2, O3 and O4 with their properties. Object O1 is in a defined relationship B1 to the object of investigation U. This defined relationship B1 is represented by a connection (edge) between the nodes representing the object O1 and the object of investigation U. The object of investigation U is also in a defined relationship B2 to the object O2. This defined relationship B2 is represented by a connection (edge) between the nodes representing the object O2 and the object of investigation U. The object of investigation U is also in a defined relationship B3 to the object O3. This defined relationship B3 is represented by a connection (edge) between the nodes representing the object O3 and the object of investigation U. The object O3 is also in a defined relationship B4 to the object O4. This defined relationship B4 is represented by a connection (edge) between the nodes representing the object O3 and the object O4.

A graph thus includes information about an object of investigation and its properties, information about other objects and their properties, and information about the relations between the object of investigation and one or more objects and, if applicable, information about the relations between one or more other objects. Each of these information items usually exists in the form of a number and/or an array of numbers. A graph can also include information about the graph itself, for example, the context. Such information is also referred to as global information; representations representing such global information are also referred to here as global representations.

The edges in a graph can be directed or non-directed. In the case of a directed edge, only one object exerts an effect on the object connected to it. In an artificial neural network that processes a graph with a direct edge, information flows only from the one object, that has an influence on the connected object, to the connected object. A directed edge can be represented by an arrow, with the arrow symbolizing the flow of information between the objects. If an edge is not symbolized by an arrow but by a straight line, information usually flows in both directions.

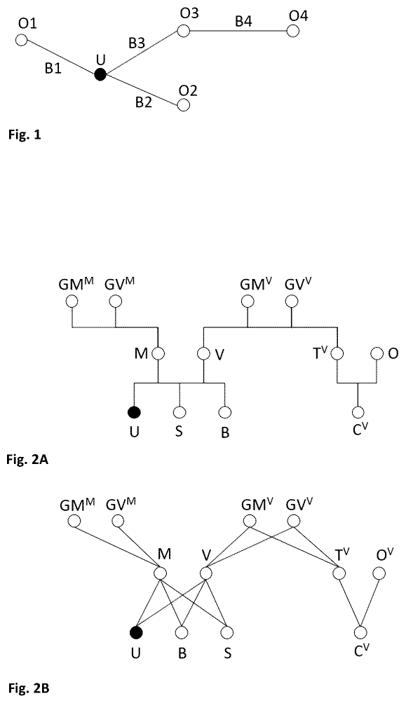

FIG. 2A and FIG. 2B show two further examples of graphs. These graphs represent a part of the family tree of an object of investigation U. The object of investigation U has a mother M and a father V. The mother M and the father V have two other children. One of these children is the sister S of the object of investigation U, the other child is the brother B of the object of investigation U. The mother M and the father V also have parents. The parents of the mother M are the maternal grandparents of the object of investigation U: a maternal grandmother GMM and a maternal grandfather GMM. The parents of the father V are the paternal grandparents of the object of investigation U: a paternal grandmother GMV and a paternal grandfather GMV. The paternal parents have a daughter. This is the sister of the father V and thus the paternal aunt TV of the object of investigation U. The paternal aunt TV together with another person, who is an uncle O of the object of investigation U, forms the parents of a person who is the paternal cousin CV of the object of investigation U.

FIG. 2A and FIG. 2B differ only in the manner in which the relationships between the persons are depicted. In FIG. 2A the relationships are shown as often customarily used with family trees. In FIG. 2B, the relationships are shown as they are customarily represented in a graph. The representation in FIG. 2A is ambiguous; it is unclear, for example, whether there is a direct connection between the object of investigation U and its sister S and/or its brother B. From FIG. 2B it can be seen that there is no direct connection in the graph between the object of investigation U and its sister S and its brother B. The connection between the object of investigation U and its sister S and its brother B is an indirect connection, which exists via the mother M and the father V. However, it should be noted that the sibling relationship between persons can also be represented in a graph by a direct connection (by an edge).

In one embodiment of the present disclosure, edges in a graph that represents a portion of a family tree represent the lineages.

As previously described, the nodes of the graph represent the object of investigation and other objects together with their respective properties.

Each node is usually itself a numerical representation of the respective object, i.e. a number and/or an array of numbers. Such a representation is also referred to as an object representation in this description.

The properties of the respective object are summarized in the object representation. For example, the properties can be summarized in the form of numbers in a property matrix (or other array of numbers). If there is a number n of objects and each object has a number f of properties, each of which can be represented by a number, the properties of all objects can be combined in a property matrix with the dimension n·f.

A preferred object representation and methods for its generation are described later in the description.

A relation between two objects in a graph can be represented by an adjacency matrix. An adjacency matrix (also known as a neighbourhood matrix) specifies which nodes of the graph are connected by an edge. An adjacency matrix can have a row and a column for each node, which for n nodes results in an n·n matrix. An entry in the i-th row and j-th column indicates whether an edge leads from the i-th to the j-th node. The number “0” can indicate that there is no edge at this point, the number “1” can indicate that an edge exists, where i and j are indices that can assume integer values in the range from 1 to n.

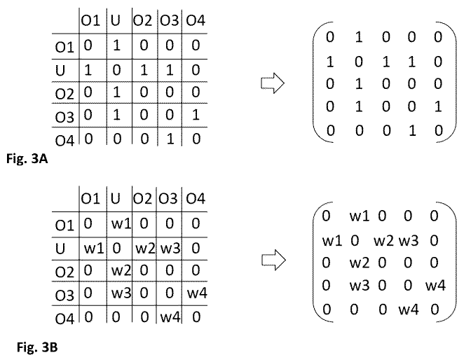

FIG. 3A shows as an example an adjacency matrix for the graph shown in FIG. 1.

In a graph, the edges can be weighted. FIG. 3B shows as an example another adjacency matrix for the graph shown in FIG. 1; the positions of the numbers “1” in the matrix shown in FIG. 3A are occupied in FIG. 3B by the weights w1, w2, w3 and w4 respectively.

Relations between objects can also be represented by an adjacency list. An adjacency list describes the connection of the edge ek between the nodes ni and nj as a tuple (i, j) in the k-th entry of a list.

In a preferred embodiment, the numerical representation representing the object of investigation with its properties and relations to one or more other objects is a graph, wherein the graph comprises an object representation for each object (including the object of investigation) and a relationship representation for each relation between two objects (including the object of investigation). The numerical representation can comprise a global representation. Each object representation, each relation representation, and the optional global representation is preferably a numerical representation, i.e. a number or an array of numbers.

In a next step, the numerical representation, which represents the object of investigation with its properties and its relations to one or more other objects, is fed to a trained machine learning model.

Such a “machine learning model” can be understood as meaning a computer-implemented data processing architecture. The model is able to receive input data and to supply output data on the basis of said input data and model parameters. The model can learn a relationship between the input data and the output data by means of training. During training, the model parameters can be adjusted so as to supply a desired output for a particular input.

During the training of such a model, the model is presented with training data from which it can learn. The trained machine-learning model is the result of the training process. Besides input data, the training data includes the correct output data (target data) that the model is intended to generate based on the input data. During training, patterns that map the input data onto the target data are identified.

In the training process, the input data of the training data are input into the model, and the model generates output data. The output data are compared with the target data. Model parameters are altered so as to reduce the differences between the output data and the target data to a (defined) minimum. The modification of model parameters in order to reduce the differences can be done using an optimization process such as a gradient process.

The differences can be quantified with the aid of a loss function. A loss function of this kind can be used to calculate a loss for a given set of output data and target data.

For example, if the output data and the target data are numerical values, the loss function can be the absolute difference between these values. In this case, a high absolute loss value can mean that one or more model parameters needs to be changed to a substantial degree.

For example, for output data in the form of vectors, difference metrics between vectors such as the mean square error, a cosine distance, a norm of the difference vector such as a Euclidean distance, a Chebyshev distance, an Lp norm of a difference vector, a weighted norm or another type of difference metric of two vectors can be chosen as the loss function.

In the case of higher-dimensional outputs, such as two-dimensional, three-dimensional or higher-dimensional outputs, an element-by-element difference metric can for example be used. Alternatively or in addition, the output data may be transformed into for example a one-dimensional vector before calculation of a loss value.

The aim of the training process may consist of altering (adjusting) the parameters of the machine-learning model so as to reduce the loss for all pairs of the training data set to a defined) minimum.

The machine learning model of the present disclosure was trained to determine an expected value for the presence and/or occurrence and/or incidence of a condition and/or event on the basis of a numerical representation supplied to the machine learning model as input data.

Essentially, a machine learning model can be trained to perform a task on the graph level, on the node level and/or on the edge level on the basis of a graph as input data.

Preferably, in the case of a graph as input data, the machine learning model is trained to perform a task at the level of the nodes, particularly preferably a task for the object of investigation.

In other words, the machine learning model is preferably trained to determine an expected value for the object of investigation. Optionally, the machine learning model can be trained to determine an expected value for the one or more other objects that are in a defined relation to the object of investigation.

The expected value indicates a probability that the condition and/or event is present and/or will occur in the (investigation) object. The term “(investigation) object” means “investigation object and/or object”.

The expected value can specify a class to which the (investigation) object is assigned. For example, a class can represent objects in which a defined condition and/or a defined event occurs. Another class can represent objects in which the defined condition does not occur and/or the defined event does not occur.

The expected value can be a probability value that indicates the probability of a defined condition occurring and/or a defined event occurring in an object. The probability value can be a number between 0 and 1 or a percentage probability.

The machine learning model has been trained on the basis of training data. The training data comprises one dataset for each of a multiplicity of reference objects. Such a dataset can be, for example, a graph. The dataset characterizes the reference object with its properties in a defined relation to other reference objects with their respective properties.

The term “multiplicity” means more than ten, preferably more than one hundred.

The term “reference” is used in this description in conjunction with the description of the training of the machine learning model. A “reference object” is an object from which data is used in training. The modifier “reference” is used to avoid clarity-related objections in the patent examination procedure, but otherwise does not have a limiting meaning.

The training data comprises, for each reference object of the multiplicity of reference objects, i) property data for the reference object and ii) relational data comprising property data for other objects that are related to the reference object, in the form of a numerical representation (for example in the form of a graph). This data acts as input data. The training data also comprises information for each reference object on the presence and/or occurrence and/or incidence of the condition and/or event in the reference object in numerical form (e.g. in the form of a number or an array of numbers). This data thus indicates whether the condition is present in the reference object and/or whether the event has occurred, and acts as target data. This information is therefore a reference expectation value for the reference object.

During training, the input data for the individual reference objects is fed (sequentially) to the machine learning model. The model is configured to generate an expected value for the reference object on the basis of the input data and model parameters.

The calculated expected value is compared with the target data (reference expected value) in order to identify deviations. The deviations between the expected value and the target data (reference expected value) can be quantified using a loss function. In an optimization procedure (e.g. a gradient procedure), the deviations can be reduced by modifying the model parameters. If the deviations have reached a defined minimum, the training can be terminated.

Training data can be obtained from clinical studies, patient records and/or publicly accessible databases (e.g. UK Biobank: https://www.ukbiobank.ac.uk/).

In a preferred embodiment, the machine learning model used to calculate the expected value is a graph network (graph neural network), or GNN) or it comprises one. Graph neural networks form a class of artificial neural networks for processing data represented as graphs.

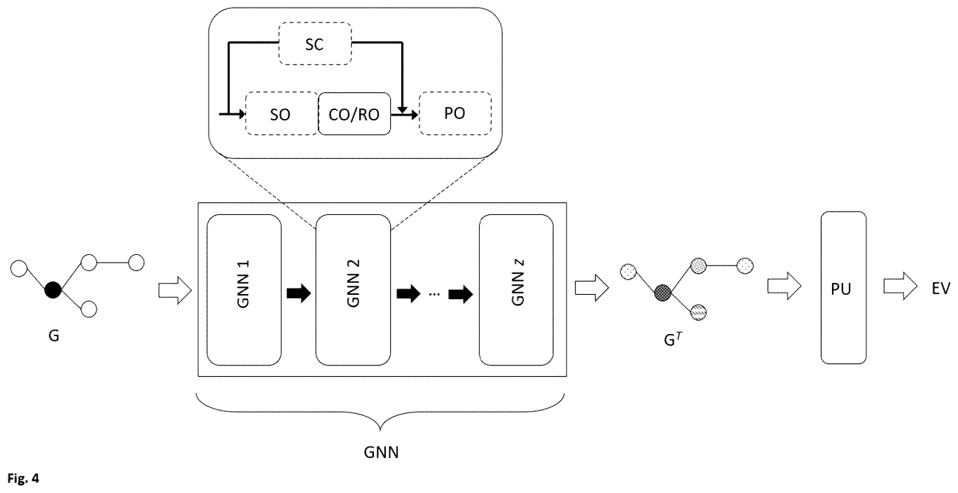

FIG. 4 schematically shows an example of a machine learning model comprising a graph neural network GNN.

A graph G can be fed to the graph network as input data. The graph network maps the graph G to a transformed graph GT. The graph network comprises a number z of blocks, which are identified in FIG. 4 with the reference numerals GNN 1, GNN 2, . . . , GNN z. Each of the blocks has operators that process information and pass it to a subsequent operator. Typical operators are a convolution operator CO, a recurrent operator RO, sampling operator SO, and a pooling operator PO. It is possible that the blocks comprise skip connections SC. Optional operators are shown in FIG. 4 as dashed boxes. The individual operators are described in more detail in scientific publications on the topic of graph networks (see e.g.: J. Zhou et al.: Graph neural networks: A review of methods and applications, AI Open 1, 2020, 57-81).

The transformed graph GT can be fed to a prediction unit PU. The prediction unit performs a task, such as a classification or regression or clustering, or other task, on the basis of the transformed graph GT.

The result of such a task can be the expected value EV.

Operators in a graph network can ensure that when processing (transforming) a graph from one block to the next, information flows from one node of the graph to other nodes of the graph. Similarly, information can flow from edges of the graph to nodes of the graph and from nodes of the graph to edges of the graph. This is called message passing and ensures that elements of the graph (nodes, edges) can influence other elements (see e.g.: J. Gilmer et al.: Neural Message Passing for Quantum Chemistry, Proceedings of the 34th International Conference on Machine Learning, Vol. 70, 2017, 1263-1272).

These information transfers cause an object representation (e.g. the representation of the object of investigation) to be affected by properties of other objects and/or by the relationship to other objects. In a training procedure, the graph network learns how properties of objects affect properties of other objects by taking into account the relations between the objects. If a machine learning model, as shown in FIG. 4, is trained end-to-end to accomplish a defined task, the graph network learns to recognize the objects, object properties and relations that are essential to the task. The graph network learns what information it needs to extract from the object properties and relations and how it needs to associate them so that the prediction unit can perform its task.

This shall be explained using an example. In the example, the object of investigation is a person. The other objects that have a defined relationship to the person are relatives of the person. The relations reflect the kinship relationships. A machine learning model is trained to determine a probability value as an expected value for the outbreak of a specific disease in the object under investigation. The machine learning model comprises a graph network. The machine learning model is trained on the basis of training data. The training data comprises a multiplicity of reference graphs. Each reference graph represents a reference person along with their characteristics in relation to relatives of the reference person with their respective characteristics. The training data also includes information for each reference person and preferably also for one or more relatives of the reference persons, whether the reference person and, if applicable, the one or more relatives of the reference person, are affected by the disease (target data). The machine learning model is trained, on the basis of the respective reference graph of a reference person (as input data), to classify the respective reference person (and, if applicable, one or more relatives of the reference person) into one of at least two classes, wherein at least one class represents reference persons (and, if applicable, relatives of the reference persons) who are not affected by the disease and at least one class represents reference persons (and, if applicable, relatives of the reference persons) who are affected by the disease. In the training, the machine learning model learns (i) what characteristics of the reference person have an influence on whether or not the disease breaks out in the reference person, and (ii) whether there may be a genetic disposition. A genetic disposition can be identified by the machine learning model, for example, by the fact that a disease is more likely in a statistical comparison to break out in a reference person if the disease has already occurred in relatives. The graph network can learn from the kinship relationships how the disposition is inherited (for example, whether the disposition is more likely to be inherited from the father or mother, whether the inheritance is recessive or dominant, whether generations are skipped and/or the like). The graph network of the machine learning model learns which characteristics of a reference person must be linked to which characteristics of one or more relatives, taking into account the kinship relationships, in order to be able to predict the onset of the disease. In other words, the graph network learns what transformation it must apply to each reference graph in order that the prediction unit of the machine learning model can predict the onset of the disease based on the transformed reference graph.

FIG. 4 shows that the transformed graph GT is supplied to the prediction unit (PU). It is possible that the transformed graph is a graph (as shown in FIG. 4). However, it is also possible that the transformed graph is an array of numbers (e.g. a matrix or a vector) that is obtained by means of a pooling procedure from the graph generated by the block GNN z. In other words, the prediction unit does not necessarily need to be supplied with a graph as input data. In particular, if a prediction is only to be made for the object of investigation, the graph resulting from the block GNN z can be changed into a form in which the information about the object of investigation, the other objects and the relations between the objects are aggregated in a numerical representation (e.g. as a matrix or a vector).

Once the machine learning model training is complete, it can be used for prediction. The trained model can be supplied with a numerical representation of a new object of investigation in relation to other objects, and the trained model calculates an expected value for the new object of investigation based on the new numerical representation. The term “new” in this case means that the numerical representation has not already been used in training the machine learning model.

The calculated expected value can be output, i.e. displayed on a screen, printed out on a printer, stored in a data storage medium and/or transmitted to a separate computer system (e.g. via a network).

As described above, multimodal data can be used to train the machine learning model and for using the machine learning trained model for prediction. This means that property data can exist in different modalities for the object of investigation, the other objects, and the reference objects.

For each object, the property data of different modalities can be combined into a single numeric representation.

This can be carried out, for example, with the aid of an autoencoder. An “autoencoder” is an artificial neural network that can be used to learn efficient data encodings in an unsupervised learning process. In general, the goal of an autoencoder is to learn a compressed representation for a data set and thus extract essential features. This allows it to be used for dimension reduction by training the network to ignore “noise”. An autoencoder comprises an encoder, a decoder, and a layer between the encoder and the decoder, which usually has a lower dimension than the input layer of the encoder and the output layer of the decoder. This layer (often referred to as bottleneck, encoding or embedding) forces the encoder to produce a compressed representation of the input data that minimizes noise and is sufficient for the decoder to reconstruct the input data.

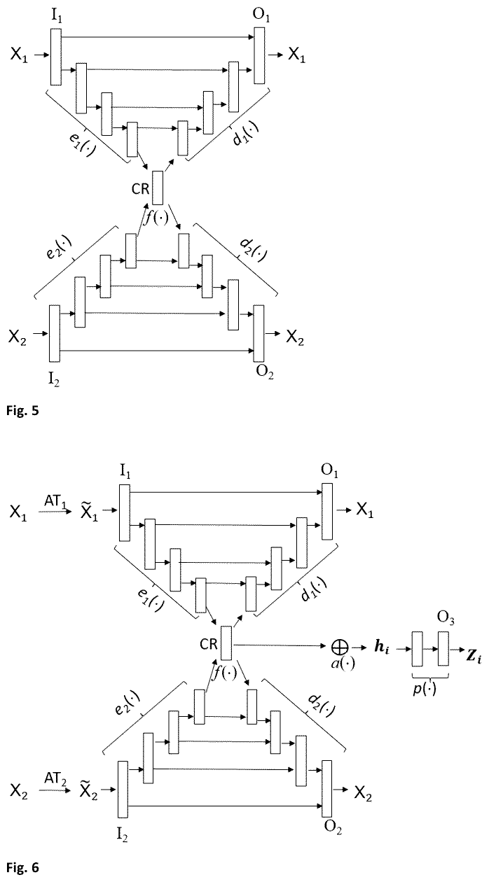

For input data in the form of different modalities, the autoencoder can comprise an encoder and a decoder for each modality, with the encoders being merged into a common layer to create a compressed representation. This is shown schematically and by way of example in FIG. 5.

The autoencoder shown in FIG. 5 comprises a first encoder e1(⋅), a first decoder d1(⋅), a second encoder e2(⋅) and a second decoder d2(⋅). The first encoder e1(⋅) has a first input layer I1 for entering data X₁ of a first modality. The second encoder e2(⋅) has a second input layer I2 for entering data X₂ of a second modality. The first encoder e1(⋅) and the second encoder e2(⋅) are merged into a common layer CR.

A fusion function f(⋅) combines the data resulting from the first encoder e1(⋅), and the data resulting from the second encoder e2(⋅). The combination of the data can be carried out, for example, by first concatenating the numerical representations e1(X₁) and e2(X₂) and then applying convolution operations to the concatenated representation. The result is a common compressed representation of the input data X₁ and X₂. Other methods for combining the data are possible.

The first decoder d1(⋅) is configured to reproduce the input data X₁ on the basis of the compressed representation and output it via the output layer O1. The second decoder d2(⋅) is configured to reproduce the input data X₂ on the basis of the compressed representation and output it via the output layer O2. The autoencoder can be trained in an unsupervised learning procedure to transform property data from different modalities of an object into a (common) representation.

FIG. 5 shows an example of an autoencoder architecture that can be used to process property data from two different modalities. If an autoencoder is to be used to process property data with three or more different modalities, such an autoencoder can process a third (and fourth, fifth, . . . ) encoder and a third (and fourth, fifth, . . . ) decoder. All encoders can be merged in the layer CR, and each decoder can reconstruct input data from a defined modality based on the basis of the common representation of the layer CR.

The known methods of data augmentation can be used to enlarge the training dataset for training the autoencoder. Augmentation techniques for images (e.g. medical images), texts and audio recordings are described, for example, in disclosures WO2022/228958A1 and WO2022/184516A1 and the references therein. When using augmented data, the augmented data can be used as input data and the non-augmented data can be used as target data. A common augmentation technique is masking. Some of the input data can be masked before the masked input data is fed to an encoder of the autoencoder. The encoder is trained to generate a compressed representation of the masked input based on the masked input data and at the same time a decoder of the autoencoder is trained to reconstruct the original, i.e. unmasked, input data based on the compressed representation. In such a case, the unmasked input data is the target data, the masked data is the input data. Through this type of masking, the autoencoder learns relationships between distinct parts of the input data that help it predict the masked (i.e. missing) data. This applies analogously to other augmentation techniques. Augmentation techniques can thus not only enlarge the training data set, but they also help to train the machine learning model in such a way that it does not simply memorize the examples presented to the model, but identifies principles and relationships in the training data. The problem of overfitting can be reduced. Thus, the trained machine learning model is better able to cope with new data, i.e. data not used in the training.

The use of augmentation techniques is illustrated exemplarily and schematically in FIG. 6. FIG. 6 shows how modified input data \tilde{X}₁ are generated from the data X₁ of the first modality by applying one or more augmentation techniques AT1. Similarly, modified input data \tilde{X}₂ is generated from the data X₂ of the second modality by applying one or more augmentation techniques AT2. The modified input data \tilde{X}₁ is fed to the first encoder e1(⋅) via the first input layer I1. The modified input data \tilde{X}₂ is fed to the second encoder e2(⋅) via the second input layer I2. The autoencoder is trained to generate a common compressed representation CR from the input data, from which the first decoder d1(⋅) reconstructs the original data X₁ and from which the second decoder d2(⋅) reconstructs the original data X₂.

FIG. 6 also shows also an extension of the learning method. The architecture shown in FIG. 6 comprises, in addition to the components already described, a further strand, which causes the architecture shown in FIG. 6, in addition to the reconstruction task, to simultaneously solve a discrimination problem (contrastive reconstruction): the model is also trained to distinguish input data X₁ originating from a first (investigation) object from input data X₂ originating from a second (investigation) object that is not identical to the first (investigation) object. In this way, the model learns to identify relationships between input data of different modalities. The strand comprises an attention-weighted pooling operation a(⋅) (see e.g. A. Radford et al.: Learning transferable visual models from natural language supervision, arXiv: 2103.00020v1) and a projection operator p(⋅), or projection head). For example, the projection head can perform a nonlinear transformation and, for example, can be a multi-layer perceptron with a hidden ReLU layer (ReLU: rectified linear unit). Details of the architecture shown in FIG. 6 and the training of such an architecture can be found, for example, in: J. Dippel, S. Vogler, S. Höhne: Towards Fine-grained Visual Representations by Combining Contrastive Learning with Image Reconstruction and Attention-weighted Pooling, arXiv: 2104.04323v2 [cs.CV], WO2022/228958A1, and WO2022/184516A1.

Once the training of the model shown in FIG. 5 or in FIG. 6 or another comparable machine learning model for generating compressed representations of multimodal input data is completed, all components with the exception of the encoders can be discarded. The encoders can be used to create compressed representations of new input data from new (investigation) objects.

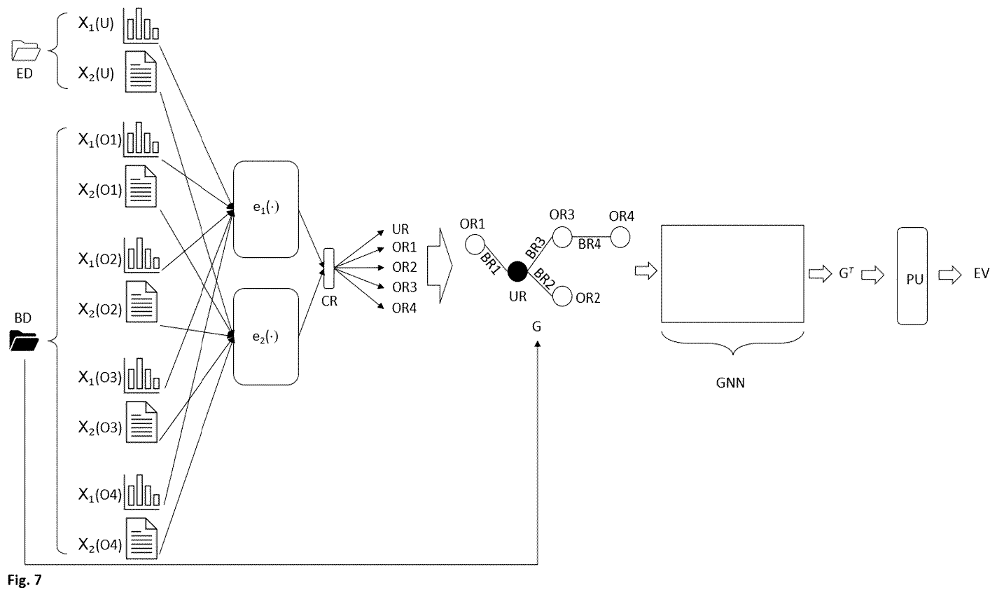

The encoders, after being trained in an autoencoder architecture, can be combined with a machine learning model as shown in FIG. 4. This is shown by way of example and in schematic form in FIG. 7.

FIG. 7 shows schematically in the form of an example how a numerical representation of this data in the form of a graph can be generated starting from property data and relational data and how an expected value can be calculated on the basis of this numerical representation.

The property data ED characterizes an investigation object U. The property data ED comprises data X₁(U) from a first modality and data X₂(U) from a second modality. The relational data BD comprises property data of four objects O1, O2, O3, and O4, which are in a defined relationship with the object of investigation U, as well as data on the relationships. The property data for object O1 comprises data X₁(O1) from a first modality and data X₂(O2) from a second modality. The property data for object O2 comprises data X₁(O2) from a first modality and data X₂(O2) from a second modality. The property data for object O3 comprises data X₁(O3) from a first modality and data X₂(O3) from a second modality. The property data for object O4 comprises data X₁(O4) from a first modality and data X₂(O4) from a second modality.

For each object (including the object of investigation), the data of the first modality is supplied to a first encoder e1(⋅) as input data, and the data of the second modality is supplied to the second encoder e2(⋅). The first encoder e1(⋅) and the second encoder e2(⋅) are merged into a layer CR, in which for each object (including the object of investigation), the data of the first modality and the data of the second modality are aggregated in a compressed representation. Thus, a compressed object representation UR is created for the object of investigation U, a compressed object representation OR1 for the object O1, a compressed object representation OR2 for the object O2, a compressed object representation OR3 for the object O3 and a compressed object representation OR4 for the object O4. Each of the compressed representations is a numerical representation, i.e. an array of numbers.

The relation representations BR1, BR2, BR3, and BR4 are generated on the basis of the data for the relationships. The relation representation BR1 characterizes the relationship between the object of investigation U and the object O1. The relation representation BR2 characterizes the relationship between the object of investigation U and the object O2. The relation representation BR3 characterizes the relationship between the object of investigation U and the object O3. The relation representation BR4 characterizes the relationship between the object O3 and the object O4. The relation representations are numerical representations, i.e. a number or an array of numbers. The relation representations can be combined into a single array, such as a (weighted) adjacency matrix or adjacency list, as described in this description. The representations of the objects (including the object of investigation) can also be combined (e.g. by scaling) in a single representation. The resulting numerical representation, which is shown in FIG. 7 as graph G, is supplied to a graph network GNN, which converts the graph G into a transformed graph GT. Based on the transformed graph GT, the prediction unit PU calculates an expected value EV. The expected value EV indicates a probability that a defined condition and/or defined event is present and/or will occur in the object of investigation U. It is also conceivable that the machine learning model shown in FIG. 7 is configured to output an expected value for one or more further objects of the objects O1, O2, O3 and O4.

As described above, the encoders e1(⋅) and e2(⋅) shown in FIG. 7 are already (pre-) trained in an autoencoder architecture. It is possible that the encoders are (further) trained in an end-to-end training procedure together with the graph network and/or the prediction unit.

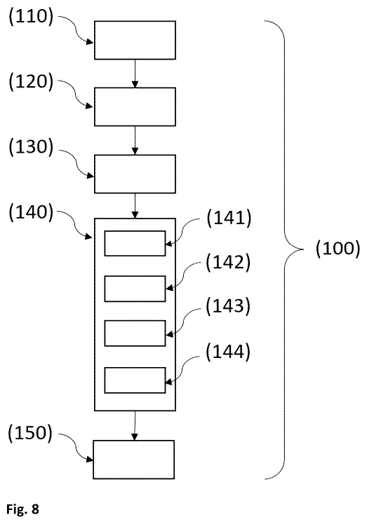

FIG. 8 schematically shows an embodiment of a method for training the machine learning model of the present disclosure. The method (100) comprises the steps of:
(110) Providing a machine learning model, wherein the machine learning model is configured to determine an expected value for the presence and/or occurrence and/or incidence of a condition and/or event on the basis of a numerical reference representation and model parameters,
(120) Providing training data, wherein the training data for each reference object of a multiplicity of reference objects comprises i) property data for the reference object and ii) relational data comprising property data for further reference objects that are related to the reference object, and data relating to relationships between the reference object and the further reference objects, and (iii) information on the presence and/or occurrence and/or incidence of the condition and/or event in the reference object,
(130) Generating a numerical reference representation for each reference object of the multiplicity of reference objects on the basis of the property data and the relational data,
(140) Training the machine-learning model, wherein the training comprises:
   for each reference object of the multiplicity of reference objects:
   (141) Inputting the numerical reference representation into the machine learning model,
   (142) Receiving an expected value as the output from the trained machine learning model, wherein the expected value indicates a probability that the condition and/or event is present and/or will occur in the reference object,
   (143) Quantifying the deviation between the expected value and the information on the presence and/or occurrence and/or incidence of the condition and/or event in the reference object,
   (144) Reducing the deviation by modifying the model parameters,
(150) Storing the trained machine learning model and/or outputting the trained machine learning model and/or transmitting the trained machine learning model to a separate computer system and/or using the machine learning model for prediction.

FIG. 9 schematically shows an embodiment of a method for using the trained machine learning model of the present disclosure for prediction. The method (200) comprises the steps of:
(210) receiving property data for an object of investigation,
(220) receiving relational data, wherein the relational data comprises property data for one or more objects that are related to the object of investigation, and data on a relationship between the one or more objects and the object of investigation,
(230) generating a numerical representation on the basis of the property data for the object of investigation and the relational data,
(240) supplying the numerical representation to a trained machine learning model, wherein the trained machine learning model was trained on the basis of training data to determine an expected value for the presence and/or occurrence and/or incidence of a condition and/or event, wherein the training data for each reference object of a multiplicity of reference objects comprises i) property data for the reference object and ii) relational data comprising property data for other reference objects that are related to the reference object, and data relating to the relationships of the reference object to the other reference objects, and iii) information on the presence and/or occurrence and/or incidence of the condition and/or event in the reference object,
(250) receiving an expected value as the output from the trained machine learning model, wherein the expected value indicates a probability that the condition and/or event is present and/or will occur in the object of investigation,
(260) outputting the expected value and/or storing the expected value and/or transmitting the expected value to a separate computer system.

The steps, methods and/or functions described in this disclosure may be performed in whole or in part by a computer system.

A “computer system” is an electronic data processing system that processes data by means of programmable calculation rules. Such a system typically comprises a “computer”, which is the unit that includes a processor for carrying out logic operations, and peripherals.

In computer technology, “peripherals” refers to all devices that are connected to the computer and are used for control of the computer and/or as input and output devices. Examples thereof are monitor (screen), printer, scanner, mouse, keyboard, drives, camera, microphone, speakers, etc. Internal ports and expansion cards are also regarded as peripherals in computer technology.

Modern computer systems are frequently divided into desktop PCs, portable PCs, laptops, notebooks, netbooks and tablet PCs, and what are called handhelds (for example smartphones); all of these systems may be used to implement the disclosed techniques.

The term “computer” should be broadly interpreted and include any type of electronic device with data processing capabilities, including, as a non-limiting example, personal computers, servers, embedded cores, communications devices, processors (e.g. Digital Signal Processor (DSP), microcontrollers, Field Programmable Gate Array (FPGA), Application Specific Integrated Circuit (ASIC), etc.) and other electronic computing devices.

The term “process”, as used above, shall include any form of calculation or manipulation or transformation of data that is represented as physical, e.g. electronic, phenomena and, for example, may occur or be stored in registers and/or memories of at least one computer or processor. The term “processor” refers to a single processing unit or a multiplicity of distributed or remote units such as this.

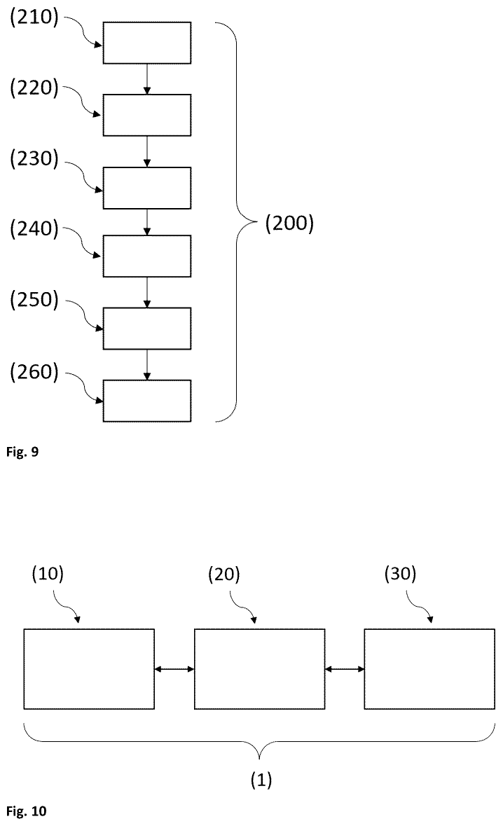

FIG. 10 shows by way of example and in schematic form an embodiment of a computer system according to the present disclosure.

The computer system (1) comprises
an input unit (10),
a control and calculation unit (20), and
an output unit (30).

The input unit (10) is used to receive data (e.g. training data, property data, relational data, information on the presence and/or occurrence and/or incidence of the condition and/or the event in one or more (reference) objects) and/or to receive commands from a user of the computer system (1).

The control and calculation unit (20) serves to control the input unit (10) and the output unit (30) and, if appropriate, other components of the computer system (1) and to coordinate the data flows and to perform calculations (e.g. with the aid of the (trained) machine learning model).

The control and calculation unit (20) is configured,
to cause the input unit (10) to receive property data for an object of investigation and relational data, wherein the relational data comprises property data for one or more objects that are related to the object of investigation, and data on a relationship between the one or more objects and the object of investigation,
to generate a numerical representation on the basis of the property data for the object of investigation and the relational data,
to supply the numerical representation to a trained machine learning model, wherein the trained machine learning model was trained on the basis of training data to determine an expected value for the presence and/or occurrence and/or incidence of a condition and/or event, wherein the training data for each reference object of a multiplicity of reference objects comprises i) property data for the reference object and ii) relational data comprising property data for other reference objects that are related to the reference object, and data relating to the relationships of the reference object to the other reference objects, and iii) information on the presence and/or occurrence and/or incidence of the condition and/or event in the reference object,
to receive an expected value from the trained machine learning model, wherein the expected value indicates a probability that the condition and/or event is present and/or will occur in the object of investigation,
to cause the output unit (30) to output and/or to store the expected value and/or to transmit it to a separate computer system.

The output unit (30) is used to output information (e.g. an expected value) to a user of the computer system (1).

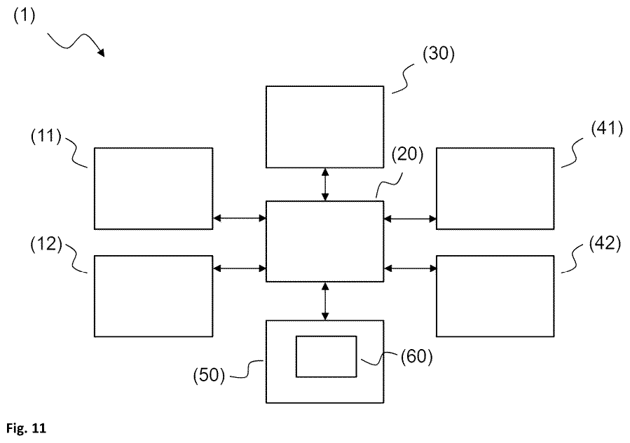

FIG. 11 shows by way of example and in schematic form a further embodiment of a computer system according to the present disclosure.

The computer system (1) comprises a processing unit (20) connected to a storage medium (50). The processing unit (20) corresponds to the control and calculation unit shown in FIG. 10.

The processing unit (20) may comprise one or more processors alone or in combination with one or more storage media. The processing unit (20) may be customary computer hardware that is able to process information such as digital images, computer programs and/or other digital information. The processing unit (20) normally consists of an arrangement of electronic circuits, some of which can be designed as an integrated circuit or as a plurality of integrated circuits connected to one another (an integrated circuit is sometimes also referred to as a “chip”). The processing unit (20) may be configured to execute computer programs that can be stored in a working memory of the processing unit (20) or in the storage medium (50) of the same or of a different computer system.

The storage medium (50) may be customary computer hardware that is able to store information such as digital images (for example representations of the examination region), data, computer programs and/or other digital information either temporarily and/or permanently. The storage medium (50) may comprise a volatile and/or non-volatile storage medium and may be fixed in place or removable. Examples of suitable storage media are RAM (random access memory), ROM (read-only memory), a hard disk, a flash memory, an exchangeable computer floppy disk, an optical disc, a magnetic tape or a combination of the aforementioned. Optical discs can include compact discs with read-only memory (CD-ROM), compact discs with read/write function (CD-R/W), DVDs, Blu-ray discs and the like.

The processing unit (20) may be connected not just to the storage medium (50), but also to one or more interfaces (11, 12, 30, 41, 42) in order to display, transmit and/or receive information. The interfaces can comprise one or more communication interfaces (41, 42) and/or one or more user interfaces (11, 12, 30). The one or more communication interfaces (41, 42) may be configured to send and/or receive information, for example to and/or from an MRI scanner, a CT scanner, an ultrasound camera, other computer systems, networks, data storage media or the like. The one or more communication interfaces (41, 42) may be configured to transmit and/or receive information via physical (wired) and/or wireless communication connections. The one or more communication interfaces (41, 42) may comprise one or more interfaces for connection to a network, for example using technologies such as mobile telephone, wifi, satellite, cable, DSL, optical fibre and/or the like. In some examples, the one or more communication interfaces (41, 42) may comprise one or more close-range communication interfaces configured to connect devices having close-range communication technologies such as NFC, RFID, Bluetooth, Bluetooth LE, ZigBee, infrared (e.g. IrDA) or the like.

The user interfaces (11, 12, 30) can comprise a display (30). A display (30) may be configured to display information to a user. Suitable examples thereof are a liquid crystal display (LCD), a light-emitting diode display (LED), a plasma display panel (PDP) or the like. The user input interface(s) (11, 12) may be wired or wireless and may be configured to receive information from a user in the computer system (1), for example for processing, storage and/or display. Suitable examples of user input interfaces (11, 12) are a microphone, an image- or video-recording device (for example a camera), a keyboard or a keypad, a joystick, a touch-sensitive surface (separate from a touchscreen or integrated therein) or the like. In some examples, the user interfaces may contain an automatic identification and data capture technology (AIDC) for machine-readable information. This can include barcodes, radiofrequency identification (RFID), magnetic strips, optical character recognition (OCR), integrated circuit cards (ICC) and the like. The user interfaces may in addition comprise one or more interfaces for communication with peripherals such as printers and the like.

One or more computer programs (60) may be stored in the storage medium (50) and executed by the processing unit (20), which is thereby programmed to fulfil the functions described in this description. The retrieving, loading and execution of instructions of the computer program (60) may take place sequentially, such that an instruction is respectively retrieved, loaded and executed. However, the retrieving, loading and/or execution may also take place in parallel.

1. A method comprising:
receiving property data (ED) for an object of investigation (U);
receiving relational data (BD), wherein the relational data (BD) comprises property data for one or more objects (O1, O2, O3, O4) that are related to the object of investigation (U), and data on a relationship (B1, B2, B3, B4) between the one or more objects (O1, O2, O3, O4) and the object of investigation (U);
generating a numerical representation (G) based on the property data (ED) for the object of investigation (U) and the relational data (BD);
supplying the numerical representation (G) to a trained machine learning model, wherein the trained machine learning model was trained using training data to determine an expected value for the presence and/or occurrence and/or incidence of a condition and/or event, wherein the training data comprises, for each reference object of a multiplicity of reference objects:
property data for the reference object,
relational data comprising property data for other reference objects that are related to the reference object and data indicating the relationships of the reference object to the other reference objects, and
information on the presence and/or occurrence and/or incidence of the condition and/or event for the reference object;
receiving an expected value (EV) from the trained machine learning model, wherein the expected value (EV) indicates a probability that the condition and/or event is present and/or will occur in the object of investigation (U); and
outputting the expected value (EV) and/or storing the expected value (EV) and/or transmitting the expected value (EV) to a separate computer system.

2. The method of claim 1, wherein the object of investigation (U) is a human being and the one or more objects (O1, O2, O3, O4) are relatives of the object of investigation (U).

3. The method of claim 1, wherein the condition is a disease and/or the event is the outbreak of a disease in the object of investigation (U).

4. The method of claim 1, wherein the property data (ED) of the object of investigation (U) and the property data for the one or more objects comprise health data, wherein the health data comprises one or more of the following data items: age, height, weight, body mass index (BMI), gender, eye colour, hair colour, skin colour, blood group, membership of an ethnic group, existing diseases and/or conditions, pre-existing diseases and/or conditions, native language, membership of a religion, marital status, nationality, date of birth, place of birth, level of education, employment, level of income, wealth, debt, creditworthiness, place of residence, living family members, history of previous illnesses, times when the diseases occurred, severity of the diseases that occurred, measures taken to cure and/or alleviate the diseases, current and/or past blood tests and/or liver tests and/or kidney tests and/or thyroid values and/or blood pressure values, resting heart rate, lung capacity, tidal volume, minute respiratory volume, internal body temperature, electrocardiogram, electroencephalogram, skin conductivity, tremor (frequency), amount and frequency of medication taken, amounts and frequency of drugs taken, such as cigarettes and/or alcohol, medical image recordings of the body and/or a part of the body, audio recordings of one or more body sounds, self-assessment data.

5. The method of claim 1, wherein the numerical representation (G) is a graph, wherein:
the object of investigation (U) is represented by a node in the graph,
each further object (O1, O2, O3, O4) of the one or more objects (O1, O2, O3, O4) is represented by a further node in the graph,
each relationship (B1, B2, B3) between the object of investigation (U) and the one or more objects (O1, O2, O3) is represented by an edge, and each relationship (B4) between any two objects (O3, O4) is also represented by an edge.

6. The method of claim 1, wherein training the machine learning model comprises:
receiving the training data,
generating a numerical representation for each reference object on the basis of the property data for the reference object and the relational data for the reference object,
inputting the numerical representation into the machine learning model,
receiving an expected value from the machine learning model,
quantifying the deviations of the expected value from the information on the presence and/or occurrence and/or incidence of the condition and/or event in the reference object, and
minimizing the deviations by modifying parameters of the machine learning model.

7. The method of claim 1, wherein the machine learning model is a graph network (GNN) or comprises such a network.

8. The method of claim 1, wherein the property data (ED) comprises data of different modalities(X₁, X₂), wherein the machine learning model for each modality comprises an encoder (e₁(⋅), e₂(⋅)), wherein the encoders (e₁(⋅), e₂(⋅)) were trained in a common autoencoder architecture to aggregate property data from different modalities in a common compressed representation (CR).

9. The method of claim 7, wherein the autoencoder architecture was trained to reconstruct property data (ED) from the common compressed representation (CR) and to distinguish property data of one object from property data of another object.

10. A computer system comprising one or more processors configured to:
receive property data (ED) for an object of investigation (U);
receive relational data (BD), wherein the relational data (BD) comprises property data for one or more objects (O1, O2, O3, O4) that are related to the object of investigation (U), and data on a relationship (B1, B2, B3, B4) between the one or more objects (O1, O2, O3, O4) and the object of investigation (U);
generate a numerical representation (G) using the property data (ED) for the object of investigation (U) and the relational data (BD);
supply the numerical representation (G) to a trained machine learning model, wherein the trained machine learning model was trained using training data to determine an expected value for the presence and/or occurrence and/or incidence of a condition and/or event, wherein, for each reference object of a multiplicity of reference objects, the training data comprises:
property data for the reference object,
relational data comprising property data for other reference objects that are related to the reference object and data indicating relationships of the reference object to the other reference objects, and
information on the presence and/or occurrence and/or incidence of the condition and/or event in the reference object;
receive an expected value (EV) as the output of the trained machine learning model, wherein the expected value (EV) indicates a probability that the condition and/or event is present and/or will occur in the object of investigation (U); and
output and/or to store the expected value (EV) and/or to transmit it to a separate computer system.

11. A non-transitory computer-readable storage medium storing software commands that, when executed by a processor of a computer system, cause the computer system to:
receive property data (ED) for an object of investigation (U);
receive relationship data (BD), wherein the relationship data (BD) comprises property data for one or more objects (O1, O2, O3, O4) that are related to the object of investigation (U), and data on a relationship measure (B1, B2, B3, B4) between the one or more objects (O1, O2, O3, O4) and the object of investigation (U);
generate a numerical representation (G) using the property data (ED) for the object of investigation (U) and the relational data (BD);
supply the numerical representation (G) to a trained machine learning model, wherein the trained machine learning model was trained using training data to determine an expected value for the presence and/or occurrence and/or incidence of a condition and/or event, wherein, for each reference object of a multiplicity of reference objects, the training data comprises:
property data for the reference object,
relational data comprising property data for other reference objects that are related to the reference object and data indicating the relationships of the reference object to the other reference objects, and
information on the presence and/or occurrence and/or incidence of the condition and/or event for the reference object;
receive an expected value (EV) as the output from the trained machine learning model, wherein the expected value (EV) indicates a probability that the condition and/or event is present and/or will occur in the object of investigation (U), and
output the expected value (EV) and/or storing the expected value (EV) and/or transmitting the expected value (EV) to a separate computer system.
